(19) United States
(12) Patent Application Publication
(10) Pub. No.: US 2005/0032756 A1
(43) Pub. Date: Feb. 10, 2005

(54) Multistage preparation for contraception based on natural estrogens

(76) Inventors: Michael Dittgen, Apolda (DE); Sabine Fricke, Jena (DE); Herbert Hoffmann, Jena (DE); Claudia Moore, Jena (DE); Michael Oettel, Jena (DE); Monika Oster Wald, Goettingen (DE)

Correspondence Address: STRIKER, STRIKER & STENBY, 103 East Neck Road, Huntington, NY, 11743, US

(21) Appl. No.: 10/891,729

(22) Filed: Jul. 15, 2004

Related U.S. Application Data
(63) Continuation-in-part of application No. 09/950,915, filed on Sep. 12, 2001.
Application No. 09/950,915 is a division of application No. 09/648,858, filed on Aug. 25, 2000, now abandoned.
Application No. 09/648,858 is a continuation of application No. 08/738,314, filed on Oct. 25, 1996, now Pat. No. 6,133,251.

(30) Foreign Application Priority Data
Oct. 28, 1995 (DE) 195 40 253.7

(51) Int. Cl. A61K 31/56

(52) U.S. Cl. 514/170

(57) ABSTRACT

A multistage preparation for contraception based on a combination of natural estrogen and synthetic gestogen is described. The preferred preparation contains estradiol valerate as the natural estrogen and dienogest or drospirenone as the synthetic gestogen. In comparison to conventional multistage preparations the multistage preparation according to the invention has a higher contraceptive reliability over the entire cycle, improved cyclic bleeding behavior and minimizes or prevents side effects, such as breast tension, headaches, depressive moods and libido changes.

### CROSS-REFERENCE

This is a continuation-in-part of U.S. patent application Ser. No. 09/950,915, filed Sep. 12, 2001, which, in turn, is a divisional of U.S. patent application Ser. No. 09/648,858, filed Aug. 25, 2000, which, in turn, is a continuation of U.S. patent application, Ser. No. 08/738, 314, filed Oct. 25, 1996, which has been allowed as U.S. Pat. No. 6,133,251, issued Oct. 17, 2000.

### BACKGROUND OF THE INVENTION

1. Field of the Invention

The present invention relates to a multistage preparation for contraception based on natural estrogen in combination with synthetic gestogen.

2. Description of the Related Art

Oral contraceptives were first marketed about 70 years ago. By continuous research the required dosages of the administered hormones have been reduced in a stepwise manner. Currently low dosage oral contraceptive preparations exist, which comprise an estrogen ingredient and a gestogen ingredient.

Multistage contraceptive preparations based on natural estrogen in combination with gestogens are known, for example from EP 0 770 388 B1, and its U.S. equivalent, U.S. Pat. No. 6,133,251. These latter preparations provided reduced intervening bleeding rates, while maintaining contraceptive effectiveness.

EP 0 770 388 B1 describes a multistage contraceptive preparation having
a first stage consisting of two to four daily dosage portions, each daily dosage portion consisting of natural estrogen as the sole effective ingredient;
a second stage consisting of two groups of daily dosage portions, each of which comprise a combination of at least one natural estrogen and at least one synthetic or natural gestogen, wherein a first group consists of 3 to 5 daily dosage portions and a second group consists of 17 to 13 daily dosage portions respectively;
a third stage consisting of two to four daily dosage portions, each daily dosage portion consisting of natural estrogen as the sole effective ingredient;
wherein the daily dosage portions of natural estrogen within each of the stages is constant but decreases from the first stage to the third stage and the amount of synthetic or natural gestogen in the second group of the second stage exceeds the amount in the first group of the second stage; and
an additional stage consisting of two daily dosage portions, each consisting of a pharmaceutically acceptable placebo.

In example 5 of EP 0 770 388 B1 the contraceptive preparation is based on a combination of estradiol valerate and dienogest. The first stage consists of three daily dosage units of 3 mg estradiol valerate as the sole active ingredient. The second stage consists of four daily dosage units of 2 mg estradiol valerate and 1 mg dienogest, with a first group of 4 daily dosage units consisting of 2 mg estradiol valerate plus 2 mg dienogest and a second group of 16 daily dosage units consisting of 2 mg estradiol valerate plus 2 mg dienogest. The third stage consists of two daily dosage units consisting of 1 mg estradiol valerate. The final stage includes 3 daily dosage units of a pharmaceutically acceptable placebo.

The serum concentration of progesterone was measured radio-immunologically to obtain the information regarding conceptive reliability. A limiting value of 4.0 ng/ml of progesterone resulted from the measurements. The average rate of intervening bleeding (break-through bleeding and spotting) dropped about 45 to 53% from the first to the last cycle.

It is also known that the contraceptive reliability of combination preparations is based on the action of both ingredients, the estrogen and the gestogen.

Furthermore it is known that the ovulation inhibiting dosage for dienogest amounts to 1.0 mg daily: see “Dienogest: Pre-Clinical and Clinical [Results for] the new Gestogen”, by A. T. Teichmann; Walter de Gruyter, Berlin/New York, p. 101 (1995). The ovulation inhibiting dosage for drospirenone amounts to 2.0 to 3.0 mg (see P. Rosenbaum, W. Schmidt, F. M. Helmerhorst, et al, “Inhibition of Ovulation by a Novel Progestogen (drospirenone)” . . . , Eur. Contracept. Reprod. Health Care 5: pp. 16-24 (2000)).

Also H.-D. Taubert and H. Kuhl (“Contraception with Hormones”, by H.-D. Taubert, et al, Georg Thieme Press, Stuttgart/New York, p. 160 (1995)) show that there is scarcely any connection or interrelation between the occurrence of intervening bleeding and lower serum concentration of estrogen, here ethinyl estradiol, or the respective gestogen.

### SUMMARY OF THE INVENTION

It is an object of the present invention to provide an agent or means for hormonal contraception based on a natural estrogen, which has a higher contraceptive reliability over the entire cycle, improved cyclic bleeding behavior and minimizes or prevents side effects, such as breast tension, headaches, depressive moods and libido changes.

According to the invention this object is attained by a multi-stage preparation for contraception comprising
a first stage consisting of two daily dosage portions, wherein each daily dosage portion in the first stage consists of 3 mg of estradiol valerate;
a second stage consisting of two groups of daily dosage portions,
   wherein a first group of daily dosage portions consists of five daily dosage portions, and each daily dosage portion in the first group consists of a combination of 2 mg estradiol valerate and at least two or three times an ovulation-inhibiting dosage of a synthetic gestogen; and
   wherein a second group of daily dosage portions consists of seventeen daily dosage positions, and each daily dosage portion in the second group consists of a combination of 2 mg estradiol valerate and at least three or four times an ovulation-inhibiting dosage of a synthetic gestogen;
a third stage consisting of two daily dosage portions, wherein each daily dosage portion in the third stage consists of 1 mg estradiol valerate; and
an additional stage consisting of two daily dosage portions of a pharmaceutically acceptable placebo.

In advantageous preferred embodiments of the invention dienogest, drospirenone or a gestogen with at least two times its known ovulation-inhibiting dosage is used as the gestogen ingredient.

The multi-stage preparation according to the invention is especially suitable for oral administration, but it is also conceivable that the method of administration could be intravaginal, parenteral, topical, rectal, intranasal, intrabuccal or sublingual.

The multi-stage preparation is made in a known way with the conventional solid or liquid carriers or diluents and contains the conventional pharmaceutical auxiliary ingredients according to the desired method of administration in a suitable dosage.

Preferably tablets, film tablets, dragees or hard gelatin tablets can be used for oral administration.

The invention will be demonstrated with a few examples hereinbelow. The contraceptive reliability, the cyclic bleeding behavior of women and the compatibility of the administration regime are demonstrated.

Contraceptive Reliability

In order to make a judgment regarding contraceptive reliability the Hoogland Score was determined. The follicle size, the estradiol level and the progesterone values were used to determine the Hoogland score. In the present case the serum concentration of progesterone was measured radio-immunologically on selected days. The number of ovulations (Hoogland score 6) and the lutinized, non-ruptured follicles (Hoogland score 5) were determined.

Cycle Stability

The cycle stability was evaluated with the help of the bleeding pattern determined for each cycle. In that connection the occurrence of intervening bleeding (spotting or break-through bleeding) is particularly interested. This sort of investigation was standardized. The evaluation of the data occurred descriptively.

Compatibility

The compatibility of the contraceptive preparation was evaluated with the help of subjective findings, such as headaches, depressive moods, breast tension, stomach complaints (nausea/vomiting), edemas and libido changes.

### EXAMPLE 1

### Invention

The following regimen was used for administration:

| | |
|---|---|
| 1 to 2 days: | 3 mg estradiol valerate/day |
| 3 to 7 days: | 2 mg estradiol valerate/day + |
| | 2 mg dienogest/day |
| 8 to 24 days: | 2 mg estradiol valerate/day + |
| | 3 mg dienogest/day |
| 25 to 26 days: | 1 mg estradiol valerate/day |
| 27 to 28 days: | placebo |

The studies were performed with 93 test subjects of ages 18 to 35 years. Administration duration amounted to three cycles each. However only the second and third cycles were observed. In the second cycle ovulation (primary target variable) took place in 3 of the 93 women (3.23%). In the third cycle ovulation occurred in 2 of the 92 women.

Thus a reliable ovulation inhibition of 96.77% as documented when the administration regime of the invention was employed. At the same time good compatibility is observed with all test subjects upon administration of the combination preparation according to the invention.

### EXAMPLE 2

### Invention

The following regimen was used for administration:

| | |
|---|---|
| 1 to 2 days: | 3 mg estradiol valerate/day |
| 3 to 7 days: | 2 mg estradiol valerate/day + |
| | 3 mg dienogest/day |
| 8 to 24 days: | 2 mg estradiol valerate/day + |
| | 4 mg dienogest/day |
| 25 to 26 days: | 1 mg estradiol valerate/day |
| 27 to 28 days: | placebo |

The studies were performed with 93 test subjects of ages 18 to 35 years. Administration duration amounted to three cycles each. However only the second and third cycles were observed. In the second cycle ovulation (primary target variable) took place in 2 of the 93 women (2.15%). In the third cycle ovulation occurred in 2 of the 92 women.

Thus a reliable ovulation inhibition of 97.85% as documented when the administration regime of the invention was employed. At the same time good compatibility is observed with all test subjects upon administration of the combination preparation according to the invention.

A sufficient ovulation inhibition of 97.85% and 96.77% can be documented for both examples. The latest tests of conventional ovulation inhibition according to R. A. Pierson, et al, “Ortho Evra/Evra versus oral contraceptives: follicular development . . . ”, Fertil. Steril. 80 (1), pp. 34-42 (2003) obtained with preparations, which have been available for a long time and have proven to be safe and reliable, attain a known percentage ovulation. In two treatment cycles, for example, ovulation occurred in 14% of the test subjects (3 out of 22) with an oral contraceptive containing a three-stage levonorgestrel preparation, ovulation occurred in 24% of the test subjects (6 out of 25) with an oral contraceptive containing a one-stage levonorgestrel preparation and ovulation occurred in 16% of the test subjects (4 out of 25) with an oral contraceptive containing a three-stage norgestimate preparation. These values clearly lie well above that of the inventive preparation, so that the preparations according to the invention provide a higher contraceptive reliability than those tested in Pierson, et al.

While the invention has been illustrated and described as embodied in a multistage preparation for contraception based on natural estrogens, it is not intended to be limited the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

1 to 4. (canceled).

5. A multistage preparation for contraception comprising
a first stage consisting of two daily dosage portions, wherein each of said daily dosage portions in said first stage consists of 3 mg of estradiol valerate;
a second stage consisting of two groups of daily dosage portions,
wherein a first group of said daily dosage portions consists of five daily dosage portions, and each of said daily dosage portions in said first group consists of a combination of 2 mg estradiol valerate and at least two or three times an ovulation inhibiting dosage of a synthetic gestogen; and
wherein a second group of said daily dosage portions consist of seventeen daily dosage positions, and each of said daily dosage portions in said second group consists of a combination of 2 mg estradiol valerate and at least three or four times an ovulation inhibiting dosage of a synthetic gestogen;
a third stage consisting of two daily dosage portions, wherein each of said daily dosage portions in said third stage consists of 1 mg estradiol valerate; and
an additional stage consisting of two daily dosage portions of a pharmaceutically acceptable placebo.

6. The multistage preparation as defined in claim 5, wherein said synthetic gestogen consists of dienogest or drospirenone.

7. The multistage preparation as defined in claim 5, where said synthetic gestogen in said first group of said daily dosage portions is present in said daily dosage portions in an amount consisting of said at least two times said ovulation inhibiting dosage.

8. The multistage preparation as defined in claim 5, wherein each of said daily dosage positions in said first group of said second stage consists of a combination of 2 mg of said estradiol valerate and 2 to 3 mg of dienogest and each of said daily dosage portions in said second group of said second stage consists of 2 mg of said estradiol valerate and from 3 to 4 mg of dienogest.
